(19) United States
(12) Patent Application Publication
(10) Pub. No.: US 2023/0165788 A1
(43) Pub. Date: Jun. 1, 2023

(54) MULTI-DRUG FORMULATIONS FOR SUBCUTANEOUS BIODEGRADABLE RESERVOIR DEVICE

(71) Applicant: Research Triangle Institute, Research Triangle Park, NC (US)

(72) Inventors: Leah Marie JOHNSON, Durham, NC (US); Linying Alice LI, Durham, NC (US); Sai Archana KROVI, Morrisville, NC (US); Ariane VAN DER STRATEN, Kensington, CA (US)

(21) Appl. No.: 17/995,542

(22) PCT Filed: Apr. 7, 2021
(86) PCT No.: PCT/US2021/026135

§ 371 Date: Oct. 5, 2022

Related U.S. Application Data
(60) Provisional application No. 63/006,163, filed on Apr. 7, 2020.

(51) Int. Cl. A61K 9/00; A61K 31/7076; A61K 31/675; A61K 45/06; A61K 47/44; A61K 47/34; A61P 31/18; A61K 31/575; A61K 31/57; A61P 15/18

(52) U.S. Cl. CPC A61K 9/0024; A61K 31/57; A61K 31/575; A61K 31/675; A61K 31/7076; A61K 45/06; A61K 47/34; A61K 47/44; A61P 15/18; A61P 31/18

(57) ABSTRACT

A reservoir device comprising an active agent formulation contained within a reservoir is described. The active agent formulation comprises more than one active agent. The reservoir is defined by a biodegradable, permeable polymer membrane. The membrane allows for diffusion of the more than one active agent of the formulation there through when positioned subcutaneously in a body of a subject.

### CROSS-REFERENCE TO RELATED APPLICATIONS

This application is an International application which claims priority to U.S. Provisional Application No. 63/006,163 filed on Apr. 7, 2020, the entire content of which is incorporated herein by reference.

### FEDERAL FUNDING LEGEND

The invention was made with support under Cooperative Agreement No: AID-OAA-A-17-00011, awarded by the United States Agency for International Development. The Government has certain rights in the invention.

### TECHNICAL FIELD

A subcutaneous biodegradable reservoir device for sustained delivery of an active agent over an extended period of time is described herein. Physical parameters of the device and active agent formulations contained therein can be selected to provide effective and sustained delivery of the active agent. In embodiments, the reservoir device may contain active agent formulations having more than one active agent.

### BACKGROUND

The need for effective biomedical interventions for preventative indications (e.g., pregnancy, infectious disease) and therapeutic needs (e.g., disease, opioid addiction) remains important worldwide. In general, end-users have persistently struggled with suboptimal adherence to daily oral or on-demand interventions. Sustained, user-independent delivery of active pharmaceutical ingredients (APIs) or active pharmaceutical agents enables users to avoid burdensome time- or event-driven regimens and bypasses many adherence challenges of user-dependent methods. Also, systemic administration, combined with long-term delivery, may significantly protect and treat many disease indications without first pass effects through the liver, which can reduce the bioavailability.

An area where improvements in biomedical intervention could prove beneficial is the global HIV epidemic. HIV Pre-Exposure Prophylaxis (PrEP) with antiretroviral (ARV) drugs is a promising biomedical strategy to address the global problem. Tenofovir-based PrEP has demonstrated successes with daily and on-demand dosing. Despite these advancements, adherence to time- or event-driven regimens for PrEP remains a struggle. Long-acting (LA) delivery of ARV drugs simplifies traditional dosing regimens for PrEP by alleviating the emotional and logistical burden of user-dependent methods. For example, a LA-injectable formulation of the integrase inhibitor, cabotegravir (CAB), is currently under investigation in two phase ⅔ HIV PrEP trials. See, HPTN083 and HPTN084. Although injectable methods are acceptable to many users and offer key advantages, such as a bi-monthly dosing regimen and discretion, drawbacks do exist. Injectable formulations cannot be removed in the event of an adverse drug-related event and the potential exists for a long plasma “tail” of sub-therapeutic drug levels.

A promising biomedical approach for LA-PrEP involves implants that reside under the skin to continuously release drug, which supports adherence over longer time periods, enables discretion of use, lowers the burden of the regimen, and remains reversible during the therapeutic duration. Polymeric implants can comprise different architectures that each has advantages for drug delivery. See Solorio, L. et al.; Yang, W.-W. et al.; and Langer, R. Reservoir-style implants involve a formulated drug core encapsulated by a rate-controlling polymeric barrier. Notable examples of implants with a core-sheath configuration include the collection of subdermal contraceptive implants: Norplant® and Jadelle® for delivery of levonorgestrel (LNG) using a rod of silicone-based polymer and Implanon® and Nexplanon® for delivery of etonogestrel (ENG) using a rod of ethylene-vinyl acetate (EVA)-based polymer. The low dosages required for subcutaneous delivery of hormonal contraceptives enable these implants to last multiple years. Reservoir-style implants have also shown utility for indications in ophthalmology.

Several implants are currently under development for HIV PrEP, with each implant system holding unique configurations and features. A subdermal, silicone implant that delivers TAF from orthogonal channels coated with polyvinyl alcohol (PVA) showed 40-days of drug delivery in beagle dogs without observed adverse events. See Gunawardana, M. et al. A non-polymeric, refillable implant designed to deliver TAF and emtricitabine (FTC) from separate devices showed sustained levels of tenofovir diphosphate (TFV-DP) in peripheral blood mononuclear cells (PBMCs) over 83 days in rhesus macaques but only 28 days for FTC-triphosphate (FTC-TP) due to the large dosing required and short plasma half-life. See Chua, C.Y.X. et al. A titanium osmotic pump system, called the Medici Drug Delivery System™, is being developed for PrEP and for type-2 diabetes. See A New Collaboration for HIV Prevention Available online. Additionally, a matrix-style PrEP implant for delivery of 4′-ethylnyl-2-fluoro-2′-dexoyadenosine (EFdA) has shown promising efficacy for HIV treatment and prevention, as demonstrated in animal models. See Barrett, S.E. et al.

Currently, there is an unmet need for a long-acting, biodegradable drug delivery implant device. If such device had zero-order drug release kinetics, it could provide a flat PK profile at a steady state. As such, when active agent was depleted from the device, only a minimal tail would be expected according to the drug’s half-life. Such technology could be used for a wide variety of therapeutics and preventatives, including small molecules and biologics.

### SUMMARY OF THE DISCLOSURE

In a first aspect of the invention, a reservoir device includes an active agent formulation contained within a reservoir. The active agent formulation comprises more than one active agent. For example, the formulation may comprise two or more active agents. The reservoir is defined by a biodegradable, permeable polymer membrane having a thickness of at least 45 µm. The membrane allows for diffusion of the more than one active agent of the formulation there through when positioned subcutaneously in a body of a subject.

Implementations may include one or more of the following features. The device where the permeable polymer membrane has a thickness of at least 45 µm. The device where the active agent formulation includes more than one active agent and an excipient.

In a second aspect of the invention, a reservoir device includes more than one active agent contained within a reservoir. The reservoir is defined by a biodegradable, permeable polymer membrane, wherein the membrane allows for diffusion of the more than one active agent there through with zero-order release kinetics for a time period of at least 60 days when positioned subcutaneously in a body of a subject.

Implementations may include one or more of the following features. The device where at least one of the more than one active agent includes tenofovir alafenamide fumarate (TAF), 4′-ethynyl-2-fluoro-2′-deoxyadenosine (EFdA), EFdA-alafenamide, levonorgestrel (LNG); etonogestrel (ENG) or combinations thereof. The device where at least one of the more than one active agent includes an antibody, a small molecule, a protein, a peptide, a hormone or a combination thereof. The device where the reservoir further contains an excipient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and other features of the disclosure are explained in the following description, taken in connection with the accompanying drawings, wherein:

FIG. 1A is a schematic representation of an exemplary drug delivery device in accordance with an aspect of the invention. The figure on the left is a perspective view of the exemplary device. The figure on the right is a top view of the exemplary device.

FIG. 1B is a labelled version of the schematic representation of FIG. 4A.

FIG. 1C is a schematic representation of another exemplary device and a photograph of the exemplary device.

FIG. 2A is a line chart showing daily EFdA release profiles of co-formulated devices containing EFdA and LNG formulations.

FIG. 2B is a line chart showing daily EFdA release profiles of co-formulated devices containing EFdA and ENG formulations.

FIG. 3A is a line chart showing daily LNG release profiles of multi-drug devices containing EFdA and LNG formulations.

FIG. 3B is a line chart showing daily ENG release profiles of multi-drug devices containing EFdA and ENG formulations.

FIG. 4A is a line chart showing daily TAF release profiles of co-formulated devices containing TAF and LNG formulations.

FIG. 4B is a line chart showing daily TAF release profiles of co-formulated devices containing TAF and ENG formulations.

FIG. 5A is a line chart showing daily LNG release profiles of multi-drug devices containing TAF and LNG formulations.

FIG. 5B is a line chart showing daily ENG release profiles of multi-drug devices containing TAF and ENG formulations.

FIG. 6A is a line chart showing daily EFDA release profiles of multi-drug devices containing EFDA and LNG formulations at different lengths.

FIG. 6B is a line chart showing daily EFDA release profiles of multi-drug devices containing EFDA and LNG formulations at different wall thicknesses.

FIG. 7A is a line chart showing daily LNG release profiles of multi-drug devices containing EFDA and LNG formulations at different lengths.

FIG. 7B is a line chart showing daily LNG release profiles of multi-drug devices containing EFDA and LNG formulations at different lengths wall thicknesses.

FIG. 8A is a line chart showing daily EFDA release profiles of multi-drug devices containing EFDA and ENG formulations at different lengths.

FIG. 8B is a line chart showing daily EFDA release profiles of multi-drug devices containing EFDA and ENG formulations at different wall thicknesses.

FIG. 9A is a line chart showing daily ENG release profiles of multi-drug devices containing EFDA and ENG formulations at different lengths.

FIG. 9B is a line chart showing daily ENG release profiles of multi-drug devices containing EFDA and ENG formulations at different wall thicknesses.

FIG. 10A is a line chart showing daily FTC and TAF release profiles of multi-drug devices containing FTC and TAF formulation (33% FTC, 33% TAF).

FIG. 10B is a line chart showing daily FTC and TAF release profiles of multi-drug devices containing FTC and TAF formulations (40% FTC, 40% TAF).

FIG. 11A is a line chart showing daily BIC and EFdA release profiles of multi-drug devices containing BIC and EFdA formulation (8% EFdA, 39.5% BIC).

FIG. 11B is a line chart showing daily BIC release profiles of multi-drug devices containing BIC and EFdA formulations.

FIG. 11C is a line chart showing daily EFdA release profiles of multi-drug devices containing BIC and EFdA formulations.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “a reservoir device” means at least one reservoir device and can include more than one reservoir device.

Unless otherwise defined, all technical terms used herein have the same meaning as commonly understood by one of ordinary skill in the art to which this disclosure belongs.

A biodegradable medical device and accompanying formulations that enable long-acting, sustained delivery of more than one active pharmaceutical ingredient (API) in a single formulation are described. The device can sustainably release more than one active agent at zero order kinetics. In embodiments, the medical device is in the form of a cylinder comprising a biodegradable polymer membrane, with the cylinder having a reservoir containing a formulation comprising at least two active agents and an excipient. The formulation can be used, in some situations, for prevention or treatment of disease. The polymer is permeable to the drug after injection into a body. The release rate of the drug is controlled by the formulation within the reservoir, the physicochemical properties of the API and excipient and the polymer thickness, the surface area of the implant. The medical device can be preferably used for long term prevention or treatment of disease or for prevention of pregnancy, or combinations of both.

The medical device is a biodegradable, zero-order implant that can accommodate more than one drug in the reservoir. Formulating more than one active agent in a single formulation (also referred to herein as co-formulating or multi-drug formulating) has benefits and advantages. For example, including more than one drug in the implant reservoir facilitates ease and scale-up during fabrication and manufacturing of the implant. Moreover, the formulation of multiple drugs can be tuned to meet targeted release rates and targeted depletion profiles (i.e., multiple drugs deplete simultaneously from the implant or at different times) as needed. Further, using a single implant with a multi-drug formulation eliminates the need for insertion of multiple implants, each with a unique drug. In embodiments, the use of multi-drug formulations results in preferred release profiles of each drug, as compared to single drug formulations. For example, ENG + TAF results in faster release rates of ENG and TAF from the implant, as compared to ENG or TAF alone.

The terms “active pharmaceutical ingredient” and “active agent” are used interchangeably throughout the present description. Moreover, the terms “co-formulation,” “multi-active agent formulation” and “multi-drug formulation” are also used interchangeably throughout the present description. The term multi-drug formulation will be understood to mean a formulation comprising more than one active agent. For example, the multi-drug formulation may comprise two, three, four, five, or more active agents. Additionally, the multi-drug formulation may also comprise one or more excipients.

The medical device has a reservoir that contains a multi-active agent formulation. The reservoir is defined by a biodegradable, permeable polymer membrane that has a thickness of at least 45 µm. In a preferred embodiment, the polymer membrane has a thickness of at least 70 µm. The membrane allows for diffusion of the more than one active agent of the formulation there through when positioned subcutaneously in a body of a subject.

The active agent formulation includes more than one active agent and an excipient. One or more of the more than one active agent can be one or a combination of a therapeutic, a preventative, a prophylactic and/or a contraceptive. In some embodiments, at least one of the active agents comprises an antibody, a small molecule, a protein, and/or a peptide. For example, in embodiments, at least one of the active agents comprises an antibody for the prevention of HIV infection. In other embodiments, at least one of the active agents comprises a nucleotide reverse transcriptase inhibitor (NRTI) for prevention of HIV infection. Exemplary active agents include Tenofovir Alafenamide Fumarate (TAF), Tenofovir (TFV), Tenofovir disoproxil fumarate, 4′-Ethynyl-2-fluoro-2′-deoxyadenosine (EFdA) or a pro drug of EFdA such as EFdA-alafenamide (or other), Abacavir, Bictegravir (BIC), Raltegravir (RTG), Dolutegravir (DTG), Levonorgestrel (LNG), Etonogestrel (ENG) Emtricitabine (FTC), Lamivudine (3TC), Tamoxifen, Tamoxifen citrate, Naltrexone hydrochloride, Naltrexone, Naloxone or combinations thereof. Not all active agents are amenable for use in the described device. Active agents having sufficient aqueous solubility and stability and dosing requirements and that are amenable to size parameters of the device are suitable for use in the described device. Moreover, in embodiments, the active agents retain a high level of purity that is both safe and efficacious to the user throughout the intended dosage duration and are not susceptible to immediate degradation caused by environmental contents (e.g., body fluids, physiological temperature). In additional embodiments, the solubility of active agents within potential excipients can range from 0.1-50 mg/mL. Whether the solubility of the active agents in the excipient enables a sufficient rate of drug release to meet therapeutic dose criteria is considered when selecting active agent/excipient pairings. For example, Elvitegravir, an integrase inhibitor used to treat HIV infection, was evaluated for use in the described device but was not selected for further development because of relatively low solubility and suboptimal potency of the drug. More particularly, the required subcutaneous dose for Elvitegravir is estimated to be ~16 mg/day. In an exemplary device, the active agent loading capacity of one device (2.5 mm x 40 mm) is about 120 mg. With these values, the implant would be depleted in a week.

Additional potential active pharmaceutical ingredients include active agents useful for various indications including, but not limited to, hormones for thyroid disorder, autoimmune disease or adrenal insufficiency, androgen replacement therapy, transgender hormone therapy, androgen deprivation therapy, growth hormone deficiency, Cushing’s syndrome, depression, use as contraceptive agents and diabetes; antibiotics; antivirals for HIV, Influenza, Rhinoviruses, Coronaviruses, Herpes, Hepatitis B, and Hepatitis C; Opioid addiction; antidepressants; antipsychotics; Attention-Deficit/Hyperactivity Disorder (ADHD); Hypertension; and Breast Cancer. Exemplary active pharmaceutical ingredients can include, without limitation, the following hormones: Levothyroxine, Thyroxine (T4), Triiodothyronine (T3), Cortisol, Dexamethasone, Testosterone, Leuprorelin, Goserelin, Triptoreline, Histrelin, Buserelin, Degarelix, cyproterone acetate, flutamide, nilutamide, bicalutamide, enzalutamide, Growth hormone, somatotropin, recombinant growth hormone, Antiglucocorticoid compounds (Mifepristone, metyrapone, ketoconazole), Insulin, Contraceptive agents such as Progestogens: desogestrel, norethisterone, etynodiol diacetate, levonorgestrel, lynestrenol, norgestrel, Estrogen, ethinylestradiol, and mestranol.

Exemplary active pharmaceutical ingredients can include, without limitation, the following antibiotics: penicillins, cephalosporins, rifamysins, lipiarmycins, quinolones, sulfonamides, macrolides, lincosamides, and tetracyclines.

Exemplary active pharmaceutical ingredients can include, without limitation, the following HIV antivirals: Integrase Inhibitors such as Dolutegravir, Elvitegravir, and Raltegravir; Nuceloside/Nucleotide reverse transcriptase inhibitors (NRTIs) such as abacavir, lamivudine, zidovudine, emtricitabine, tenofovir disoproxil fumarate, tenofovir alafenamide, EFdA, didanosine, stavudine, and zalcitabine; Non-nucleoside reverse transcriptase inhibitors (NNRTIs) such as efavirenz, etravirine, nevirapine, rilpivirine, and delavidine mesylate; Protease inhibitors such as atazanavir, cobicistat, lopinavir, ritonavir, darunavir, fosamprenavir, tipranavir, nelfinavir, indinavir, saquinavir, and amprenavir; Entry Inhibitors such as enfuviride; CCR5 antagonists such as maraviroc, and vicriviroc; and P4503A inhibitors such as cobicistat and ritonavir. Exemplary active pharmaceutical ingredients can further include, without limitation, the following influenza antivirals: Amantadine, Umifenovir, Moroxydine, Nitazoxanide, oseltamivir, peramivir, rimantadine, zanamivir; the following Herpes antivirals: Acyclovir, edoxudine, famciclovir, foscarnet, inosine pranobex, idoxuridine, penciclovir, trifluridine, valaciclovir, vidarabine; the following Hepatitis B antivirals: Adefovir, entecavir, pegylated interferon alfa-2a; and the following Hepatitis C antivirals: Sofosbuvir, simeprevir, ledipasvir, daclatasvir, velpatasvir, telaprevir, and taribavirin. Exemplary active pharmaceutical ingredients can further include, without limitation, remdesivir, hydroxychloroquine, chloroquine, and azithromycin. Exemplary APIs can further include, without limitation, corticosteroids, including prednisone, prednisolone, methylprednisolone, beclometasone, betamethasone, dexamethasone, fluocortolone, halometasone and mometasone.

Exemplary active pharmaceutical ingredients can include, without limitation, the following active agents for use with opioid addiction: Methadone, buprenorphine, naltrexone, naloxone, nalmefene, nalorphine, nalorphine dinicotinate, levallorphan, samidorphan, dezocine, nalbuphrine, pentazocine, phenazocine, and butophanol. Exemplary active pharmaceutical ingredients can include, without limitation, the following antidepressants and antipsychotics: Citalopram, Escitalopram, Fluoxetine, Fluvoxamine, Paroxetine, Sertraline, Desvenlafaxine, Duloxetine, Levomilnacipran, Milnacipran, Venlafaxine, Vilazodone, Vortioxetine, Trazodone,, Atomoxetine, Reboxetine, Teniloxazine, Viloxazine, Bipropion, Amitriptyline, Amitriptylinoxide, Clomipramine, Desipramine, Dibenzepin, Dimetacrine, Dosulepin, Doxepin, Imipramine, Lofepramine, Melitracen, Nitroxazepine, Nortriptyline, Noxiptiline, Opipramol, Pipofezine, Protriptyline, Trimipramine, Tetracyclic antidepressants, Amoxapine, Maprotiline, Mianserin, Mirtazapine, Setiptiline, Amisulpride, Aripiprazole, Brexpiprazole, Lurasidone, Olanzapine, Quetiapine, Risperidone, Buspirone, Lithium, and Modafinil. Exemplary active pharmaceutical ingredients can include, without limitation, the following agents for ADHD: Adderall XR, Concerta, Dexedrine, Evekeo, Focalin XR, Quillivant XR, Ritalin, Strattera, and Vyvanse. Exemplary active pharmaceutical ingredients can include, without limitation, the following agents for Hypertension: Beta-blockers such as cebutolol, atenolol, betaxolol, bisoprolol, bisoprolol/hydrochlorothiazide, metoprolol tartrate, metoprolol succinate, nadolol, pindolol, propranolol, solotol, timolol; Angiotensin converting enzyme inhibitors (ACE inhibitors) such as benazepril, captopril, enalapril, fosinopril, lisinopril, moexipril, perindopril, quinapril, ramipril, trandolapril; and Angiotensin-receptor blockers (ARBs) such as candesartan, eprosartan, irbesartan, losartan, telmisartan, valsartan. Exemplary active pharmaceutical ingredients can include, without limitation, the following agents for Breast Cancer: Tamoxifen, anastrozole, exemestane, letrozole, fulvestrant, toremifene. Exemplary active pharmaceutical ingredients can include, without limitation, the following agents: Rintatolimod for Chronic fatigue syndrome, Cidofovir, Fomivirsen for cytomegalovirus retinitis, Metisazone for smallpox, pleconaril for picornavirus respiratory infection, ribavirin for Hepatitis C or viral hemorrhagic fevers, and valganciclovir for cytomegalovirus CMV infection.

An excipient can be mixed with the more than one active agent to form the active agent formulation, and thus, is also contained within the reservoir. Exemplary excipients include, but are not limited to, castor oil, sesame oil, oleic acid, polyethylene glycol, ethyl oleate, propylene glycol, glycerol, cottonseed oil, polysorbate 80, synperonic PE/L or combinations thereof. Criteria for down-selection of the excipients include the stability (e.g., chemical purity) and compatibility (e.g., physical mixing properties) of the active agent formulation, and support of targeted release kinetics. As used herein, the stability of a component (active or excipient) means that the component retains its original chemical structure and biological activity after exposure to an environmental condition. For example, a component may have a chemical stability greater than 90%, as determined by HPLC-UVVIS analysis. Additional potential excipients include, for example, polyethylene glycol 300 (PEG 300), PEG 400, PEG 600, PEG40, α-cyclodextrin, P-cyclodextrin, and γ-cyclodextrin.

The choice of excipient to use in a multi-drug formulation with active agents can affect the release rate and release profile of the active agents. For example, the solubility of the particular active agents in an excipient can affect the release rate and profile of the active agents. In some embodiments, an excipient with higher solubility for the active agents can show a faster release rate. Moreover, the choice of excipient may have little to no effect on the release profile. For example, in formulations wherein a relatively small amount of excipient is used, the excipient may have little to no effect on the release profile.

Additionally, the formulation or concentration ratio of active agent or agents to excipient can affect the release profile of the active agent. In embodiments, it is desirable to find a maximum ratio or optimal ratio of active agent(s) to excipient that maximizes loading capacity of active agents in the device while maintaining a zero-order release profile. When the ratio of active agents to excipient is above the maximum ratio, the release profile may not be a linear, zero-order release profile. However, the release profile may transition to a linear, zero-order release profile over time, as active agents are released from the device. A device having an active agent formulation with a ratio of active agents to excipient that is below the maximum ratio may provide a zero-order release profile. All other parameters being the same (for example, excipient type, active agent, device size, and membrane thickness), the device with the lower ratio of drugs to excipient has fewer active agents than a device having the maximum ratio and thus will likely have a shorter active agent release duration than the device with the maximum ratio.

Moreover, the properties and characteristics of a particular active agent or agents and a particular excipient can determine the formulation ratio that is ideal for a particular application. Accordingly, the formulation ratio for a single active agent may be different depending on the excipient that is used. Moreover, the formulation ratio for one active agent in a multi-agent formulation may be different depending on the second (or subsequent) active agent in the multi-agent formulation.

Two processes are involved in the controlled release of an active agent or agents: 1) Dissolution of the active agent (e.g., TAF) within an excipient, and 2) Diffusion of the active agent solution through the polymer membrane.

With the dissolution process, particles of active agent are continuously being dissolved in the excipient solution. The Noyce-Whitney equation can be used to describe the dissolution process:

$$\frac{d m}{d t} = A \frac{{D}_{s} \left({C}_{s} - {C}_{b}\right)}{h}$$

In the Noyce-Whitney equation, dm/dt is the dissolution rate, A is the surface area of the interface between the substance and the solvent, Dₛ is the diffusion coefficient within the excipient, h is the thickness of the diffusion layer, Cₛ is the saturation concentration of the substance within the solvent, and C_{b} is the mass concentration of the substance in the bulk of the solvent.

With the diffusion process, the active agent (e.g., TAF) first partitions into the membrane and then diffuses to the other side of the membrane. Fick’s First Law of Diffusion can be used to describe the diffusion process:

$$J = - {D}_{m} \frac{d φ}{d x}$$

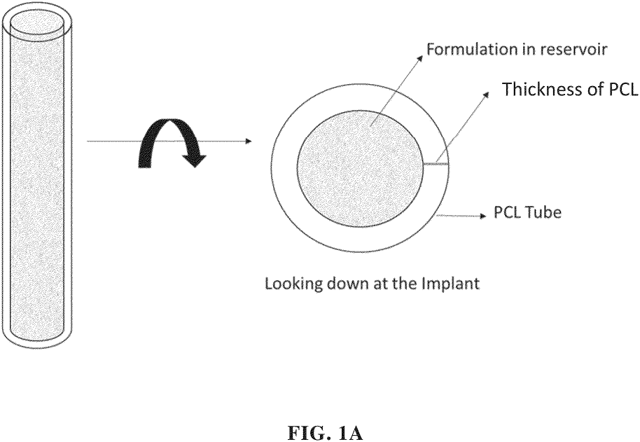

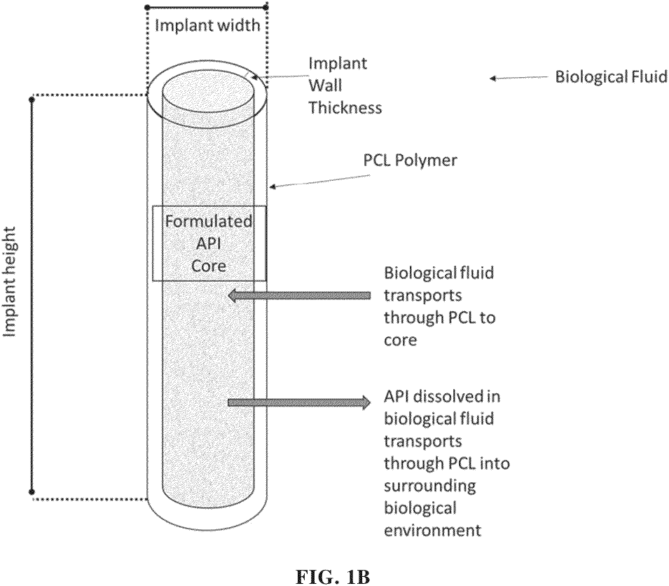

In Fick’s first law of diffusion, J is diffusion rate or the amount of drug released from the membrane per unit area per unit time, Dm is diffusion coefficient through the membrane, φ is concentration, and x is length. FIG. 1 is a labelled, schematic representation of a drug delivery device.

According to Fick’s first law of diffusion, when the reservoir is saturated, a constant concentration gradient dφ/dx is maintained in the membrane, so the rate for drug flux J is constant and zero order release is achieved. The constant release rate for the diffusion-controlled process can be calculated according to the modified diffusion equation:

$$J = {D}_{m} K \frac{{C}_{s}}{L}$$

In the modified equation, J is the amount of drug released from the membrane per unit area per unit time (mg/day/mm²), Dm is diffusion coefficient through the membrane, K is partition coefficient, Cs is the saturation concentration of the substance within the excipient, L is thickness of the PCL membrane.

When the dissolution rate is greater than the diffusion rate, the release rate is membrane controlled and the release profile is linear. In contrast, when the dissolution rate is less than the diffusion rate, the release rate is dissolution limited or controlled and the release profile is nonlinear.

The active agent formulation can include additional components. For example, antioxidant components (e.g., α-tocopherol, retinyl palmitate, selenium, Vitamin A, Vitamin C, cysteine, methionine, citric acid, sodium citrate, methyl paraben, and propyl paraben), buffering agents and hydrophile lipophile balance (HLB) modifiers can be included in the formulation. Exemplary buffering agents and HLB modifier include, but are not limited to, sodium citrate, dibasic potassium phosphate, sodium succinate, meglumine, glycine, tromethamine, Labrafac WL 1349 (HLB 1), Compritol 888 (HLB 1), Labrafil M2130 (HLB 9) and Gelot 64 (HLB 10). Binders can also be used in the formulation including sugar alcohols (e.g., xylitol, sorbitol, mannitol), polysaccharides (e.g., starches, cellulose, hydroxypropyl cellulose), or disaccharides (e.g., sucrose, lactose). One of ordinary skill in the art will understand that additional suitable excipient components may be included as appropriate and/or as needed.

The biodegradable, permeable polymer membrane also affects the release kinetics of the active agent. For example, the thickness of the membrane affects the release rate of the more than one active agent. As the thickness of the membrane increases, the release rate of the active agents decreases. In exemplary embodiments, the membrane can have a thickness ranging from about 45 µm to about 500 µm. For example, the membrane may have a thickness of 45 µm, 50 µm, 60 µm, 70 µm, 80 µm, 90 µm, 100 µm, 110 µm, 120 µm, 130 µm, 140 µm, 150 µm, 160 µm, 170 µm, 180 µm, 190 µm, 200 µm, 210 µm, 220 µm, 230 µm, 240 µm or 250 µm, 260 µm, 270 µm, 280 µm, 290 µm, 300 µm, 320 µm, 340 µm, 360 µm, 380 µm, 400 µm, 420 µm, 440 µm, 460 µm, 480 µm, or 500 µm.

The polymer membrane can comprise homopolymers, blends of more than one homopolymer, block co-polymers, or combinations thereof. Configurations of the co-polymers can include random, linear block co-polymers, and star-shaped block co-polymers. A non-limiting example of a block co-polymer is ABA, where A is a crystallizable block and B is an amorphous block. A non-limiting example of a star-shaped block co-polymer includes the combination of Poly-ε-caprolactone and Poly-valerolactone. Exemplary embodiments of the device may include one or more of the following polymers: Poly-ε-caprolactone, Poly(ε-caprolactone-co-ε-decalactone), Polyglycolic acid, Polylactic acid, Poly(glycolic-co-lactic) acid, Polydioxanone, Polyvalerolactone, Poly(3-hydroxyvalerate), Poly(3-hydroxylbutyrate), Polytartronic acid, and Poly(β-malonic acid).

The molecular weight of the polymer can affect the release rate of the active agents. For example, release rates of active agents from the implant can be tuned using polymers of different starting molecular weights. Moreover, polymer compositions that include binary polymer blends offer the ability to further tailor biodegradation rates, API release rates, and mechanical properties. The membrane of the device may comprise homopolymers. As used herein, “homopolymer” means a polymer chain comprising a single monomer. Homopolymers can be different molecular weights. Non-limiting examples of homopolymers include poly-ε-caprolactone (PCL), poly(L-lactide), poly(D-lactide), poly(D,L-lactide), polyglycolide (PGA), polyacrylic acid, polydioxanone (PDO), poly(valerolactone), poly(3-hydroxyvalerate), poly(3-hydroxylbutyrate) (3-PHB), poly(4-hydroxylbutyrate) (4-PHB), polyhydroxyvalerate (PHV), polytartronic acid, poly(D,L-methylethylglycolic acid), poly(dimethylglycolic acid), poly (D,L-ethylglycolic acid), and poly(P-malonic acid) or combinations thereof. In certain embodiments, blends of two homopolymers are used.

In certain embodiments, the membrane of the implant may comprise co-polymers. Co-polymers can comprise different connectivity including block co-polymers, graft co-polymers, random co-polymers, alternating co-polymers, star co-polymers, and periodic co-polymers. Nonlimiting examples of co-polymers include poly(L-lactide-co-D,L-lactide), poly(L-lactide-co-D-lactide), poly(L-lactide-co-glycolide), poly(L-lactide-co-ε-caprolactone), poly(D,L-lactide-co-ε-caprolactone), poly(D,L-lactide-co-glycolide), poly(glycolide-co-ε-caprolactone), poly(ε-caprolactone-co-D,L-ε-decalactone), polylactide-block-poly(ε-caprolactone-co-ε-decalactone)-block-poly(lactide), poly(ethylene glycol-co-ε-caprolactone), poly-ε-caprolactone-co-polyethylene glycol, poly(3-hydroxylbutyrate-co-3-hydroxylvalerate), poly(ethylene glycol-co-lactide), or combinations thereof.

For example, the membranes may comprise polycaprolactone (PCL) at a number average molecular weight ranging from 15,000 to 140,000 Da. In some embodiments, a higher molecular weight PCL (e.g., 80 kDa) results in a faster release rate of active agent, whereas a lower molecular weight PCL (e.g., 45 kDa) results in a slower release rate of active agent. In embodiments, implants can be fabricated from PCL tubes with MW of approximately 50 kDa (PC08), 72 kDa (PC12), 106 kDa (PC17), 130 kDa (PC31), and >130 kDa (PC41).

In embodiments, the implant is designed to biodegrade within the body after the active agents are depleted. The biodegradable polymer (e.g., PCL) can be tuned to meet the requisite biodegradation properties (that is, to optimize the time between depletion of active agents and complete polymer biodegradation). For example, biodegradation can be tuned by selecting targeted molecular weights of a homopolymer (e.g., PCL of 45 kDa or 80 kdA or blends) or by using co-polymers, as listed above. The polymer membrane has an initial molecular weight at implantation. In embodiments, the polymer membrane is configured such that the molecular weight of the membrane is reduced to a molecular weight ranging from 10 kDa to 2 kDa after the active agents are depleted from the device. For example, the molecular weight may be reduced to a molecular weight ranging from about 8 kDa to about 3 kDa after the drugs are depleted from the device. Without being bound by theory, it is believed that PCL undergoes biodegradation via bulk mode hydrolysis. For example, substantial loss of weight and fragmentation of polymer can occur at about 5 kDa MW, with intracellular bioresorption taking place at about 3 kDa MW. In embodiments, the polymer membrane can be configured such that it undergoes fragmentation at a time ranging from about 1 month to about 6 months after the active agents are depleted from the device. In this regard, exemplary embodiments having 80 kDa MW PCL films have shown an extended rate of biodegradation, typically on the order of >24 months. Further description is provided by the examples below.

The polymer membrane can comprise a blend of homopolymers with the same composition but different molecular weights (MW). For example, the polymer membrane could comprise a blend of one or more of PC08, PC12, PC31, PC41, and PC17, where each homopolymer is PCL, but the average molecular weight of each is different. The polymer membrane may comprise a blend of homopolymers, where each homopolymer has a different composition and a different molecular weight. For example, the polymer membrane could comprise a blend of PCL and PLA. The polymer membrane may comprise co-polymers, blends of co-polymers, or blends of homopolymers and co-polymers.

Additionally, the composition, molecular weight and thickness of the membrane affect the biodegradation rate of the device. The device comprised of the biodegradable polymer is placed subcutaneously in a subject. It releases active agents for an intended dosage duration. The device is designed to lose integrity due to biodegradation at time proximate to but after availability of the active agents. That is, parameters of the polymer membrane can be chosen to enable the device to maintain integrity for at least as long as the intended dosage duration of the active agents in the device.

In embodiments, the device structure maintains integrity for a time period of about 3 months to about 2 years. For example, the device may be effective for active agent delivery for 3 months, 6 months, 9 months, 12 months, 15 months, 18 months, 21 months or 24 months. In embodiments, the device may be effective for delivery of active agents for at least 3 months, at least 6 months, at least 9 months, at least 12 months, at least 15 months, at least 18 months, at least 21 months, at least 24 months or up to 3 months, up to 6 months, up to 9 months, up to 12 months, up to 15 months, up to 18 months, up to 21 months, or up to 24 months.

The device is designed for subcutaneous implantation, which simplifies administration but constrains the size of the device and the reservoir. In embodiments, the device can have a cylindrical shape, such as a cylinder with a length ranging from about 10 mm to about 50 mm and a width (or diameter) ranging from about 1 mm to about 3 mm. Moreover, the device can be fabricated by extrusion of an FDA-approved biodegradable polymer to generate a fillable tube. The tube can then be ultrasonically welded, or heat sealed to enclose the reservoir to contain the active agents.

In an embodiment, the device has a cylindrical shape and comprises a biodegradable polymer film that contains a reservoir of active agent formulation for prevention or treatment of disease.

Characteristics, including desired release rate, drug-loading capacity, geometry, dimensions, and biodegradation rate can be considered when determining which form of the device to use. For example, the target release rate and loading capacity of the device can depend on the class and potency of the active agents. Wall thickness, surface area, and formulation can be adjusted to achieve desired characteristics. Maximum number of drugs in the device reservoir (drug loading capacity) is a limiting factor to consider for maximum daily dose of agents. In exemplary embodiments, the polymer in the device can be designed to degrade in-vivo following depletion of the active agents. The biodegradation timeframe of the polymer depends on the starting molecular weight (MW) of the polymer.

Release profiles of the active agents are affected, among other things, by the properties of the polymer used for the device, including surface area, thickness, and molecular weight (which affects crystallinity). These properties can be tuned to provide desired dosing for the active agent’s delivery and desired time frame for polymer bioresorption.

An exemplary embodiment of the implant device can include a subcutaneous biodegradable implant device for multipurpose prevention technology (MPT) for HIV and pregnancy prevention. The implant can be used to simultaneously deliver combinations of biologics, such as antibodies, and small molecules. An exemplary implant device uses a semi-crystalline aliphatic polyester, PCL, pioneered by Pitt et al. in the 1980s (G. Pitt, et al.) and largely neglected for nearly 20 years (Woodruff, M.A. et al.). Renewed appeal for PCL has surfaced in light of biomedical applications, including tissue engineering and drug delivery that demand materials with long-term functionality, mechanical integrity, biocompatibility, and capacity for biodegradation and bioresorption. PCL is currently used in FDA-approved products for root canal fillings (Resilon®) and sutures (Monocryl®) and was previously explored for use as a 1-year contraceptive implant (Capronor®). In terms of HIV PrEP, PCL implants can advantageously offer long-acting delivery of ARVs, while also enabling bioresorption at the end of the implant lifetime. A biodegradable implant can benefit health care systems by eliminating the need for a clinic visit, whereby a minor surgical procedure would be required to remove the implant. For this device, reversibility and retrievability are available throughout the duration of treatment.

In embodiments of the device, the release rate of the active agent is controlled by various parameters, including, but not limited to, the formulation within the reservoir, the physicochemical properties of the active agents and the polymer film, the surface area of the device, and the thickness of the polymer film. In preferred embodiments, the reservoir device can be used for relatively long-term prevention or treatment of disease or for prevention of pregnancy, or combinations of both.

Advantageously, the biodegradable reservoir device has a zero-order release profile. Moreover, the reservoir device has additional beneficial attributes. For example, the device is subcutaneous; can release more than one active agent for various periods of time including about 3 months to about 2 years; is removable within the window of drug delivery; can be used for zero-order release of multiple active agents; and can be tuned based on various considerations, including, for example: (1) active agents; (2) excipient composition and concentration (e.g., ratio of excipient to active agent); (3) polymer membrane thickness, molecular weight, composition and crystallinity; and (4) device surface area. The device can provide long acting, zero-order release of more than one active agent. Moreover, the release kinetics are tunable to meet different dosing requirements.

The reservoir device is designed for subcutaneous implantation, which simplifies administration thereby facilitating access in resource-limited settings. Moreover, the biodegradable device can alleviate the need for an extra clinic visit to remove the implant after active agent depletion. However, because active agent is delivered through a device rather than a gel or nanosuspension, the device can be removed or retrieved throughout the duration of use. This feature can be beneficial in clinical situations requiring swift removal (e.g., product-related serious adverse event). Additionally, the reservoir device can simultaneously deliver combinations of biologics, such as antibodies, and/or small molecules.

The reservoir device can be designed for controlled release of a wide range of therapeutic and preventive active pharmaceutical ingredients (also referred to herein as active agents). Unlike other sustained release technologies, membrane-controlled devices can be functionally tuned to achieve zero-order release kinetics thereby attaining a relatively flat drug release profile and a relatively tight concentration range over several weeks to months to potentially years.

Polymer properties and drug formulations affect the release rate of active agents through polymer membranes. Thus, it is important to keep these properties in mind when designing the described reservoir devices in order to achieve zero-order release kinetics. The present disclosure describes different reservoir devices, including devices having different properties, such as differences in molecular weight, different active agents, different excipients, different formulation concentrations, and differences in membrane thickness, ultimately tuning release kinetics according to required dosage and duration.

A schematic representation of an embodiment of the device is shown in FIGS. 1A and 1B. As shown, a polymer membrane encapsulates a reservoir of formulated active agents. Passage of biological fluid into the implant solubilizes the active agents, whereupon the active agents are controllably released from the device. Release kinetics of the device are affected by the properties of the polymer membrane. In this embodiment, the device is a flexible, permeable polymer membrane cylinder filled with active agents and excipient.

As shown in FIGS. 1A and 1B, the device comprises active agents and excipient contained in a reservoir defined by a polymer membrane enclosed by heat sealing or by an ultrasonic weld. The membrane is permeable to the active agents after implantation of the device into a body of a subject. The polymer membrane allows for diffusion of the active agents through the polymer membrane when positioned subcutaneously in a body of a subject.

FIG. 1C provides a schematic representation of another exemplary device. In FIG. 1C, the device includes a formulated drug core (A) encapsulated by a rate-controlling PCL membrane (B). The device is end-sealed using PCL material (C) for trocar compatibility.

The device in FIG. 1C is a reservoir-style PCL implant that can deliver co-formulated active agents at sustained, zero-order release kinetics. Once inserted subcutaneously, biological fluid from the surrounding environment transports through the PCL membrane into the reservoir to solubilize the active agents, whereupon the active agents then transport passively through the PCL membrane and exit the implant. Without being bound by theory, it is believed that as an aliphatic polyester, PCL undergoes bulk hydrolysis through random chain scission as water permeates through the polymer. However, biodegradation of PCL is slow and can require years (e.g., 1-2 years) for complete bioresorption, depending on the starting MW. Because bulk erosion of PCL is slow, the faster process of drug delivery is decoupled from biodegradation, enabling zero-order release profiles of drug from the implant. At this zero-order release profile, the daily drug delivery rates can be controlled by various parameters: surface area of the device, thickness of the device wall, polymer properties, and drug formulation.

In some embodiments, the device can be manufactured by folding a polymer membrane over to define tubular-shaped cavity, depositing active agent formulation into the cavity, and applying an ultrasonic force or heat sealing to the membrane to create a seal that contains the active agent formulation within the tubular-shaped reservoir. The membrane allows for diffusion of active agent there through when the device is positioned subcutaneously in a body of a subject.

In other embodiments, the implant is fabricated using the following steps: (1) Extrusion of a polymer tube that comprises a hollow cylinder of polymer. The thickness of the wall can vary and in certain embodiments can measure between 50 µm and 400 µm. An exemplary wall thickness of the tube is between 200 µm and 300 µm. An exemplary outer diameter (OD) is 2.5 mm. An exemplary length of tube is 40 mm. The exemplary OD and length permit use of the implant with commercially available trocars. (2) A formulation of at least two drugs is loaded into the hollow portion of the tube. The drug formulation is produced by combining at least two drugs with an excipient. In non-limiting examples, the formulation is loaded into the tube via syringe. Exemplary excipients include castor oil, sesame oil, PEG, glycerol, and ethyl oleate. (3) Then ends of the tube are sealed to secure the drug formulation within the reservoir. In non-limiting examples, the sealing occurs by application of heat to the polymer to melt the polymer into a capped end piece.

The ability to use more than one drug within the reservoir can eliminate complications with fabrication. For example, use of a multi-drug formulation can eliminate the need for segmented implants, where each segment contains a unique active agent formulation. Segmented devices have weak points at the segmented junctions, which could be prone to mechanical failure and leakage. Using a segmented device also reduces the total available drug load in an implant because the segmented walls (i.e., portion of the polymer that forms the segment) occupy valuable space in the total length of the small implant (e.g., 40 mm). In another example, use of a multi-drug formulation eliminates the need to deliver two separate implants to the patient, where each individual implant contains a single API.

Simultaneous, long-acting delivery of more than one drug is valuable for multiple reasons. For example, it enables simultaneously prevention of infectious disease and pregnancy. The need of women for effective biomedical interventions for prevention of infectious disease and contraception is critical. Systemic administration of drugs that prevent infectious disease, combined with long-term delivery, may significantly protect a wider variety of routes of infection, including vaginal, rectal, and parenteral. Similarly, there is an unmet need for a long-acting biodegradable implant for a contraceptive method. Implants that are simple, acceptable, and accessible hold great potential for significant impacts in public health. Women can receive dual protection discreetly, even if their stated intention is to address just one health need, because of pressures from their sociocultural context (e.g., HIV stigma) or relationships.

Simultaneous, long-acting delivery of more than one drug is valuable for controlling the release rate of drugs from the implant. In certain embodiments, implants with co-formulations of ARV and contraceptive hormone result in release rates that differ from implants that contain a formulation of single active agent. In one non-limiting example, an implant containing a co-formulation of ENG and TAF results in a higher release rate of both drugs, compared to implants with a single formulation of ENG or TAF.

Additionally, the implants described herein enable multi-antiretroviral drugs for HIV treatment. Highly Active Antiretroviral Therapy (HAART) typically requires the dosing of multiple ARVs that target different stages of the life cycle of HIV. HAART regimens often require a person to take multiple pills daily, which is burdensome and prone to lessened adherence. The ability to deliver multiple drugs from a single implant via a long-acting sustained delivery implant would improve adherence and reduce burden for HIV positive individuals. A long-acting reduction in viral loads would also lessen the chance of transmission of HIV (i.e., treatment for prevention).

The implants described herein enable administration of multiple drugs to treat different classes of infectious disease. Individuals with comorbidities that comprise multiple infectious diseases would benefit from a single implant that delivers multiple drugs. Examples include coinfection with combinations of two or more, HIV, Hepatitis (A, B, or C), TB, gonorrhea, and malaria.

The implants described herein enable simultaneous treatment of substance use disorders and HIV Individuals that struggle with substance use disorder and are also HIV positive (or at high risk for acquiring HIV) would benefit from an implant that delivers ARVs and drugs to treat opioid addiction including methadone, buprenorphine, naltrexone, naloxone and combinations.

Methods are provided herein in the EXAMPLES for evaluating devices comprising PCL membranes that meet mechanical properties required for device insertion and utilization using commercially available injection systems. The dimensions and geometry of the devices have been tuned to accommodate injector systems, such as trocar used for the Jadelle contraceptive implant for hormonal therapy.

### EXAMPLES

### Example 1. Fabrication of a Biodegradable Reserovir-Style Device With Multi-Drug Formulations

Extruded polycaprolactone (PCL) tubes were cut to a length of 40 mm and heat sealed at one end. A multi-drug formulation was prepared by mixing the first drug, the second drug, and one excipient. The mixture was placed in a mortar and pestle and ground for 10 minutes. The multi-drug formulation was loaded into a syringe and the syringe was used to fill a PCL tube that contained a single heat-sealed end. After filling the PCL tube with the multi-drug formulation, the second end of the implant was heat sealed.

### Example 2. In Vitro Demonstration of Zero-Order Kinetics From Multi-Drug Formulations and Effect of Ratio of Active Agents to Excipient On Release of Active Agents From Device

Testing was performed to evaluate multi-drug formulations comprising antiretroviral and hormone for HIV prevention and contraception. Exemplary dual-drug combinations included 1) 4′-Ethynyl-2-fluoro-2′-deoxyadenosine (EFdA) mixed with levonorgestrel (LNG) at different ratios, 2) EFdA mixed with etonogestrel (ENG) at different ratios; 3) tenofovir alafenamide (TAF) mixed with LNG at different ratios, and 4) TAF mixed with ENG at different ratios.

TABLE 1

EXEMPLARY MULTI-DRUG FORMULATIONS

| | Antiretroviral | Hormone | Excipient | Amounts |
|---|---|---|---|---|
| 1 | EFdA | LNG | Castor oil or sesame oil | Varying ratios |
| 2 | EFdA | ENG | Castor oil or sesame oil | Varying ratios |
| 3 | TAF | LNG | Sesame oil | Varying ratios |
| 4 | TAF | ENG | Sesame oil | Varying ratios |

In this example, the active agent combinations were formulated with one excipient (e.g., castor oil, sesame oil). Exemplary excipients may include, but are not limited to, castor oil, sesame oil, oleic acid, polyethylene glycol, ethyl oleate, propylene glycol, glycerol, cottonseed oil, polysorbate 80, synperonic PE/L or combinations thereof.

In Vitro Testing of Exemplary Multi-Drug Formulations containing EFdA -Hormone - Excipient

Exemplary multi-drug formulations included EFdA, hormone, and excipient at concentrations of 50/35/25 wt.% or 50/25/25 wt.%, respectively. The formulations were contained within 100 µm extruded tubes fabricated with PCL of 93 KDa MW sourced from Corbion (PC-17 polymer). The implants had a length of 15 mm and an outer diameter of 2.5 mm. The implants were incubated in 200 mL of 1X PBS (pH- 7.4) at 37° C. Drug quantity released in media was measured via the HPLC-UV instrument three times per week during which the implants were transferred to fresh buffer to maintain sink conditions.

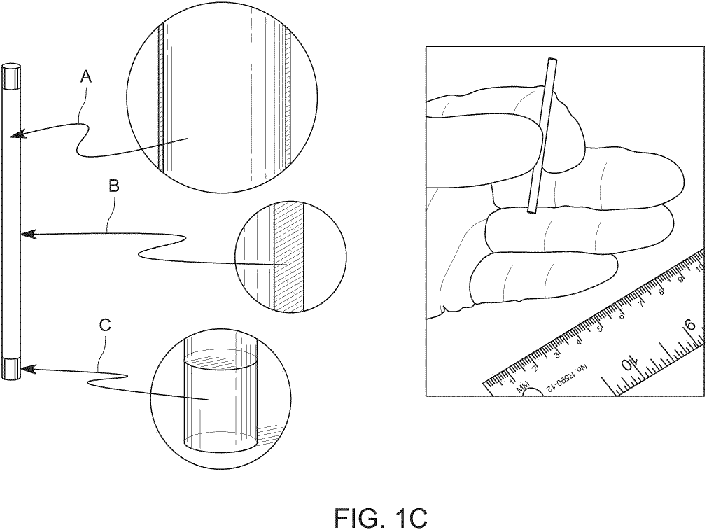

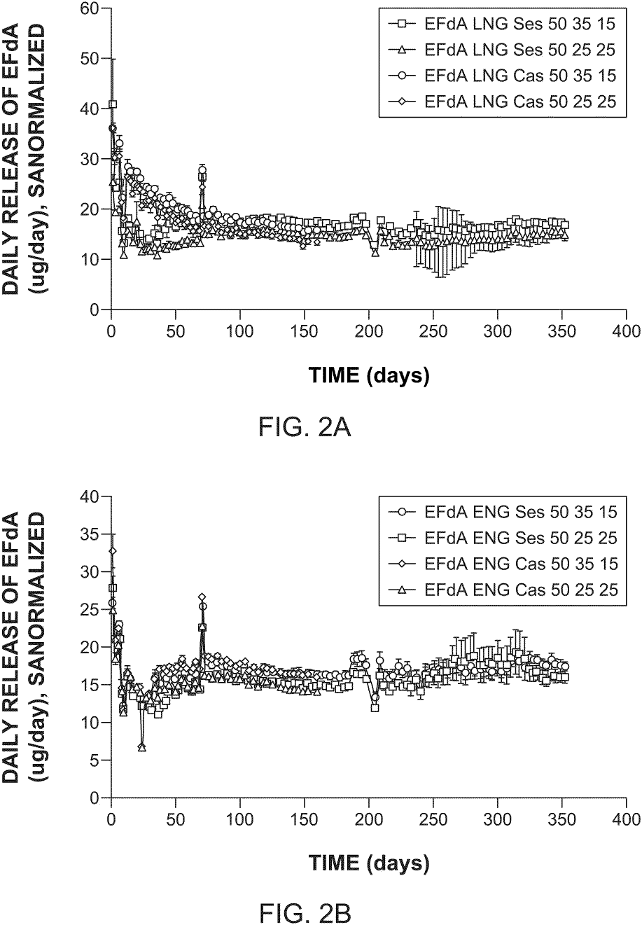

Linear release profiles were observed for devices comprising EFdA co-formulated with hormones (LNG and ENG) and excipients (castor oil and sesame oil) at two different concentration ratios (50/35/15 wt.% and 50/25/25 wt.%). FIGS. 2A and 2B are line charts showing daily release profiles of EFdA from co-formulated devices over 300 days.

The linear release profiles indicate a membrane-controlled release process for the co-formulated devices containing EFdA and hormones. Although EFdA/LNG/Castor oil devices demonstrated a higher initial release rate than the EFdA/LNG/Sesame oil devices, no significant differences in release rates were observed between the implants after 350 days. Without being bound by theory, this observation may be due to a relatively low concentration of excipients incorporated into the co-formulation and low EFdA release rates.

The release rates of the devices were normalized to the surface area of a 10 mm-long implant. Thus, calculations can be performed to enable the targeted release rates to be achieved using an implant with a longer length. The approximate release rates (based on the normalized calculation) of the co-formulated EFdA devices are shown in Table 2.

The average EFdA release rate of the multi-drug formulations was around 16.4 ± 1.3 µg/day, which is comparable to release rates of EFdA alone devices (19.6 ± 5.0 µg /day) with PC17 extruded tubes having 100 µm wall thickness. The results of this example indicate that formulating EFdA with ENG or LNG does not appear to significantly affect the release rate of EFdA.

TABLE 2

APPROXIMATE EFDA RELEASE RATE OF CO-FORMULATED DEVICES CONTAINING EFDA, HORMONE (LNG OR ENG), AND EXCIPIENT FORMULATIONS.

| API | Excipient | Excipient Wt.% | ARV Wt.% | Hormone Wt.% | Release rate of EFdA (µg/day/cm) |
|---|---|---|---|---|---|
| EFdA, LNG | Sesame Oil | 50 | 35 | 15 | 17.0 ± 3.0 |
| | | 50 | 25 | 25 | 14.4 ± 1.9 |
| | Castor Oil | 50 | 35 | 15 | 18.8 ± 4.6 |
| | | 50 | 25 | 25 | 16.5 ± 4.8 |
| EFdA, ENG | Sesame Oil | 50 | 35 | 15 | 17.8 ± 2.0 |
| | | 50 | 25 | 25 | 16.6 ± 2.4 |
| | Castor Oil | 50 | 35 | 15 | 18.3 ± 3.5 |
| | | 50 | 25 | 25 | 16.0 ± 2.5 |

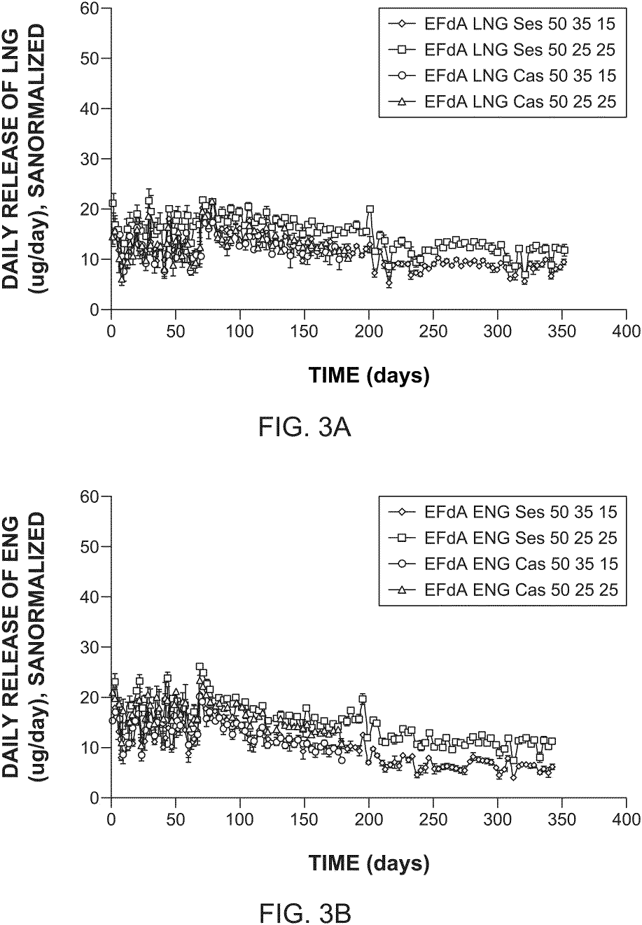

FIGS. 3A and 3B are line charts showing daily hormone release profiles (LNG or ENG) of multi-drug devices containing EFdA and hormone (LNG or ENG) formulations. As shown, the co-formulated EFdA/hormone devices exhibited sustained zero-order release of LNG and ENG. Similarly, the same constant release rate was observed for the multi-drug formulations at different drug-excipient ratios. This result confirmed that the membrane-controlled release process was achieved for the hormones.

Additionally, it was also observed that the release profiles of devices formulated with castor oil or sesame oil overlapped. This result indicates that the excipients did not significantly affect the release rate of hormones.

Table 3 shows the approximate hormone release rates of the EFdA/hormone/excipient implants (normalized to the surface area of a 10 mm-long implant). As can be seen, the release of ENG was higher than the LNG release rate. This result aligned with historical release rate data for ENG and LNG for single active agent devices. However, the co-formulated devices released ENG at a lower rate (15.2 ± 2.5 µg/day) as compared to devices containing only ENG and excipient (51.5 ± 19.2 µg/day), while the LNG release rate of the multi-drug formulation (14.4 ± 1.5 µg/day) was similar to that of devices containing only LNG and excipient (~ 22.7 ± 7.2 µg/day).

TABLE 3

AVERAGE HORMONE RELEASE RATE OF CO-FORMULATED DEVICES CONTAINING EFDA AND HORMONE (LNG OR ENG) FORMULATIONS

| API | Excipient | Excipient wt.% | ARV Wt.% | Hormone Wt.% | Release rate of hormone (µg /day/cm) |
|---|---|---|---|---|---|
| EFdA, LNG | Sesame Oil | 50 | 35 | 15 | 12.6 ± 3.4 |
| | | 50 | 25 | 25 | 16.6 ± 3.6 |
| | Castor Oil | 50 | 35 | 15 | 13.8 ± 2.5 |
| | | 50 | 25 | 25 | 14.4 ± 3.1 |
| EFdA, ENG | Sesame Oil | 50 | 35 | 15 | 11.4 ± 4.2 |
| | | 50 | 25 | 25 | 16.6 ± 4.2 |
| | Castor Oil | 50 | 35 | 15 | 14.7 ± 3.2 |
| | | 50 | 25 | 25 | 18.1 ± 3.2 |

In Vitro Testing of Exemplary Multi-Drug Formulations containing TAF-Hormone - Excipient

For exemplary implants, TAF was co-formulated with hormone (ENG or LNG) and excipient at different concentration ratios: 33/33/33 wt.%, 50/35/15 wt.%, or 50/25/25 wt.%.

To produce the exemplary implants, the mixtures were ground in a mortar and pestle and loaded into 100 µm PCL extruded tubes comprising Corbion PC-17. The implants were incubated in 150 ml of 1X PBS (pH 7.4) at 37° C. TAF and hormone concentrations released in media over time were measured via UV-Vis, and HPLC-UV, respectively. The devices were transferred to fresh buffer three times per week to maintain sink conditions.

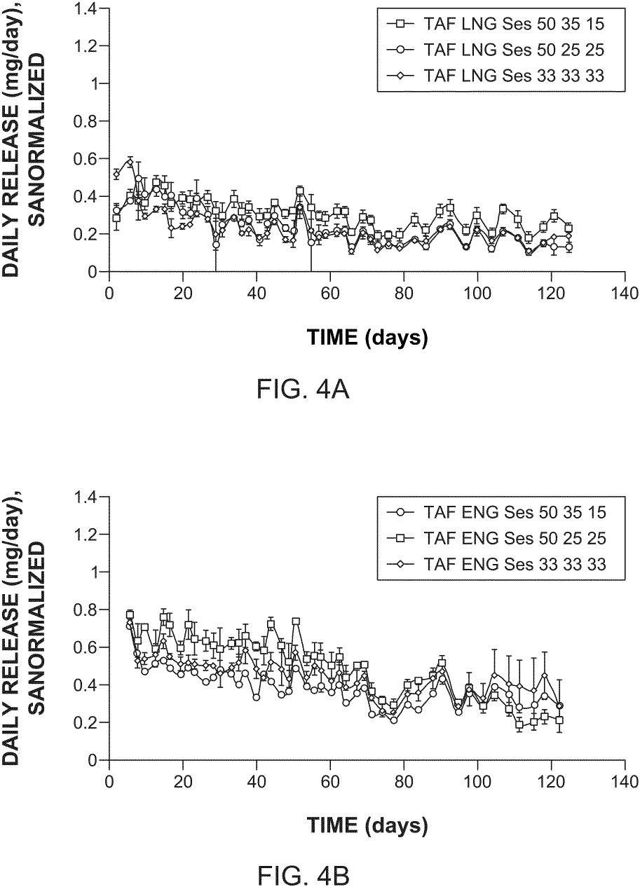

FIGS. 4A and 4B are line charts showing the daily release profiles of TAF from various TAF/hormone/excipient formulations. The implants had a length of 40 mm and an outer diameter of 2.5 mm. As can be seen, the co-formulated TAF/hormone/excipient devices exhibited linear release profiles with a constant release rate over 120 days.

The approximate daily release rates of TAF formulated with hormones and excipients at various concentrations are shown in Table 4. The TAF release rates of the devices were normalized to the surface area of a 40 mm-long implant. Unlike EFdA, the release rate of TAF was affected by the presence of hormones. For example, TAF/ENG/excipient devices released at 0.25 ± 0.04 mg of TAF per day, which is lower than the daily release rate of TAF/excipient formulation (0.35 ± 0.09 mg/day) within 100 µm PCL tubes comprising PC-17. In contrast, TAF/LNG/excipient devices exhibited a higher release rate (i.e., 0.44 ± 0.04 mg/day) than devices containing formulations having only TAF as an active agent. Without being bound by theory, the higher release rate of TAF from devices containing TAF co-formulated with LNG (TAF/LNG/excipient devices) may be attributed to a faster release of LNG from the devices, which resulted in a higher rate of water ingress.

TABLE 4

AVERAGE TAF RELEASE RATE FROM CO-FORMULATED DEVICES CONTAINING TAF AND HORMONE (LNG OR ENG) FORMULATIONS

| API | Excipient | Excipient wt.% | ARV wt.% | Hormone wt.% | Release rate of TAF (mg/day) |
|---|---|---|---|---|---|
| TAF, ENG | Sesame Oil | 33 | 33 | 33 | 0.31 ± 0.07 |
| | | 50 | 35 | 15 | 0.24 ± 0.10 |
| | | 50 | 25 | 25 | 0.23 ± 0.09 |
| TAF, LNG | Sesame Oil | 33 | 33 | 33 | 0.39 ± 0.10 |
| | | 50 | 35 | 15 | 0.51 ± 0.16 |
| | | 50 | 25 | 25 | 0.45 ± 0.10 |

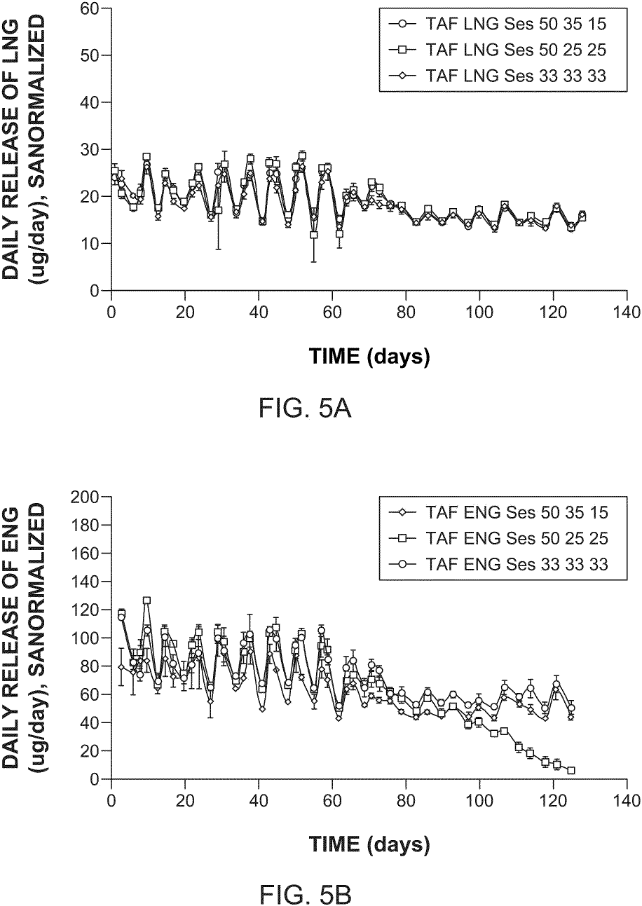

Testing also showed a sustained zero-order release of hormones from the co-formulated TAF/hormone/excipient devices. FIGS. 5A and 5B are line charts showing the daily hormone release profiles of multi-drug devices containing TAF and hormone (LNG or ENG) formulations. FIGS. 5A and 5B show the same constant hormone release rate for devices comprising TAF/hormone/excipient formulations at varying concentrations. This result indicates that the hormones were released from the devices via a diffusion-controlled process.

The approximate release rates of LNG or ENG from the multi-drug formulations are provided in Table 5. The release rates were normalized to the surface area of a 10 mm-long implant. As can be seen, the release rate of ENG was significantly higher than the LNG release rate, which is consistent with historical data for single active agent formulation devices. The average release rate of LNG from the multi-drug formulation (17.4 ± 0.4 µg/day) was also similar to that from single-drug LNG formulation (~ 22.7 ± 7.2 µg/day).

The implants containing TAF/ENG/excipient formulations exhibited an ENG release rate of 63.5 ± 4.2 µg/day in comparison to an ENG release rate of 51.5 ± 19.2 µg/day for implants containing an ENG/excipient alone formulation. These results suggest that the release rates of ENG are influenced by the presence of TAF.

TABLE 5

AVERAGE HORMONE RELEASE RATE OF CO-FORMULATED DEVICES CONTAINING TAF AND HORMONE (LNG OR ENG) FORMULATIONS

| API | Excipient | Excipient wt.% | ARV wt.% | Hormone wt.% | Release rate of hormone (µg/day/cm) |
|---|---|---|---|---|---|
| TAF, LNG | Sesame Oil | 33 | 33 | 33 | 17.6 ± 3.9 |
| | | 50 | 35 | 15 | 17.9 ± 4.4 |
| | | 50 | 25 | 25 | 16.5 ± 4.2 |
| TAF, ENG | Sesame Oil | 33 | 33 | 33 | 58.6 ± 14.4 |
| | | 50 | 35 | 15 | 63.2 ± 27.0 |
| | | 50 | 25 | 25 | 68.8 ± 16.4 |

In summary, the multi-drug formulations provided simultaneous, sustained release of ARV and hormone from a single drug reservoir over 300 days. The ARV/hormone/excipient formulations with varying drug excipient ratios exhibited the same constant release rate when membrane-controlled release was achieved. The data suggested that the release rates of EFdA and LNG are not affected by co-formulation with another active agent, whereas the release rates of TAF and ENG were altered by the presence of the other active agent in the co-formulation. In addition, unlike previously tested EFdA/excipient alone formulations, excipients did not seem to play a significant role in dictating the release rates of the EFdA/hormone/excipient co-formulations. Without being bound by theory, this result may be attributed to a relatively low concentration of excipients within the co-formulation.

In Vitro Testing of Exemplary Multi-Drug Formulations containing EFdA -LNG - sesame oil at different lengths and wall thicknesses

Exemplary lead multi-drug formulations were down selected for further evaluation, which included EFdA, LNG, and sesame oil at a concentration of 50/25/25 wt.% and EFdA, ENG, and sesame oil at a concentration of 50/35/15 wt.%. To identify the parameters that dictate the release rates of co-formulated devices, the down-selected formulations were contained within extruded tubes comprising PC-17 polymer at different wall thicknesses and implant lengths. The implants were incubated in 200 mL of 1X PBS (pH- 7.4) at 37° C. Drug quantity released in media was measured via the HPLC-UV instrument twice per week during which the implants were transferred to fresh buffer to maintain sink conditions.

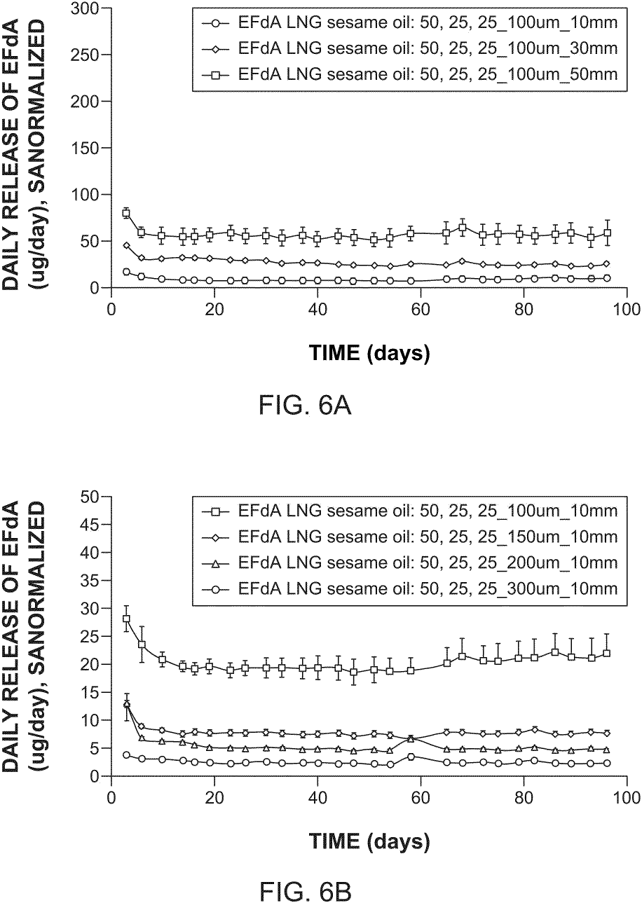

To evaluate the relationship between release rates and the surface area of the extruded PCL tubes, implants were fabricated with three different surface areas as generated by varying the implant length: 10, 30, and 50 mm. All devices comprised PC-17 with a wall thickness of 100 um, and a formulation of EFdA, LNG, and sesame oil at a concentration of 50/25/25 wt.%. FIG. 6A are line charts showing linear release profiles of EFdA from co-formulated devices over 90 days at implant length of 10, 30, and 50 mm. Similar to single formulations, a higher surface area results in a higher release rate of EFdA from the implant. This confirms that the daily release rates of co-formulated devices scale with the surface area of the implant, supporting the mechanism of membrane-controlled release from these implants.

The thickness of the implant walls was another attribute that affected release rates of EFdA. FIG. 6B are line charts showing linear release profiles of EFdA from co-formulated devices over 90 days at wall thicknesses of 100, 150, 200, and 300 µm. Similar to single formulations, the release rate of the EFdA is inversely correlated with the wall thickness of PCL walls. The release rates of EFdA decrease from 19.5 ± 1.8 µg/day to 2.3 ± 0.4 µg/day as the wall thickness of the implant increases from 100 µm to 300 µm. Thus, the release rates of the co-formulated implants are tunable via the wall thickness of the PCL.

The approximate release rates of the co-formulated EFdA devices are shown in Table 6. Similarly, the average EFdA release rate of the multi-drug formulations is comparable to the release rates of EFdA alone devices with PC17 extruded tubes. The results of this example further confirm that formulating EFdA with LNG does not appear to significantly affect the release rate of EFdA.

TABLE 6

THE AVERAGE EFDA RELEASE RATE OF CO-FORMULATED DEVICES CONTAINING EFDA AND LNG FORMULATIONS.

| Formulations | Length of devices | Wall thickness | Release rate of EFdA (µg/day) |
|---|---|---|---|
| EFdA LNG sesame oil: 50, 25, 25 | 10 | 100 | 19.5 ± 1.8 |
| | 30 | 100 | 42.2 ± 4.1 |
| | 50 | 100 | 73.7 ± 4.5 |
| | 10 | 150 | 7.4 ± 1.0 |
| | 10 | 200 | 5.1 ± 1.5 |
| | 10 | 300 | 2.3 ± 0.4 |

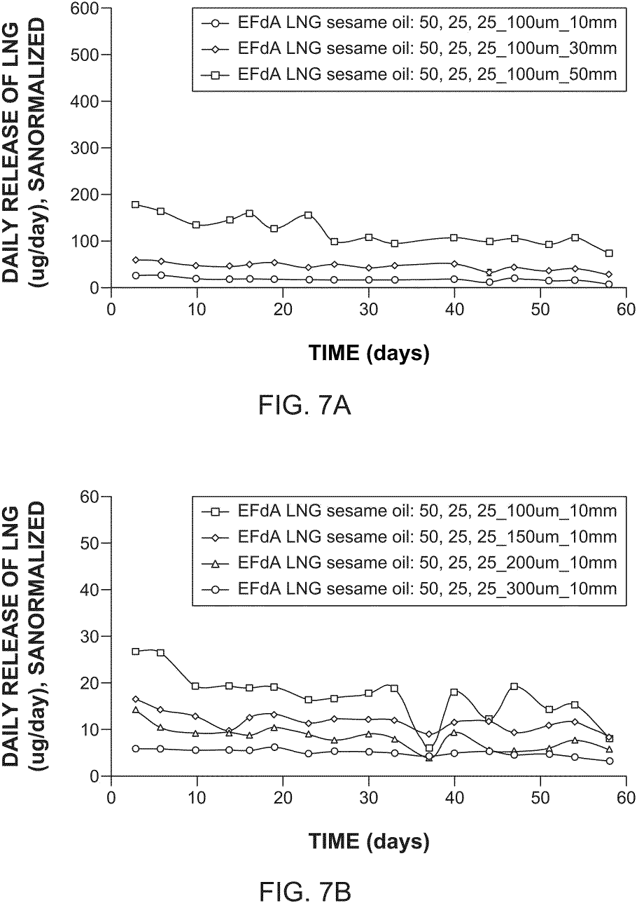

The release of LNG from the EFdA/LNG/sesame oil coformulation was also assessed. FIG. 7A line charts showing daily LNG release profiles of multi-drug devices containing EFdA LNG sesame oil formulations at different lengths. As shown, all the co-formulated EFdA/LNG devices exhibited sustained zero-order release of LNG over 50 days. Similarly, the release rates of LNG are proportional to the surface area of the implants: higher release rates were achieved for devices with larger surface areas. This result also confirmed that the membrane-controlled release process was achieved for the hormones.

FIG. 7B shows the daily release profile of multi-drug devices containing EFdA LNG sesame oil formulations at different wall thicknesses. Similarly, the release rates of LNG decrease from 18.5 ± 4.0 µg/day to 5.3 ± 0.7 µg/day as the wall thickness increases from 100 um to 300 µm. This result confirms that the release rates of LNG are also inversely proportional to the wall thickness of the PCL implants.

Table 7 shows the approximate hormone release rates of the EFdA/LNG/sesame oil implants. The LNG release rate of the multi-drug formulation was comparable to that of devices containing only LNG and excipient, which is well-aligned with previous data. This confirms the previous observation that co-formulating LNG with ARVs does not affect the release of LNG.

TABLE 7

THE AVERAGE LNG RELEASE RATE OF CO-FORMULATED DEVICES CONTAINING EFDA AND HORMONE (LNG OR ENG) FORMULATIONS.

| Formulations | Length of devices | Wall thickness | Release rate of LNG (µg/day) |
|---|---|---|---|
| EFdA LNG sesame oil: 50, 25, 25 | 10 | 100 | 18.5 ± 4.0 |
| | 30 | 100 | 48.7 ± 7.9 |
| | 50 | 100 | 89.3 ± 19.2 |
| | 10 | 150 | 12.2 ± 1.9 |
| | 10 | 200 | 8.4 ± 2.1 |
| | 10 | 300 | 5.3 ± 0.7 |

For exemplary implants, EFdA was also co-formulated with ENG and sesame oil at a concentration ratio of 50/35/15 wt.%. Devices at different wall thicknesses and different lengths were also fabricated to assess the effect of implant dimension on the release rates of the implant.

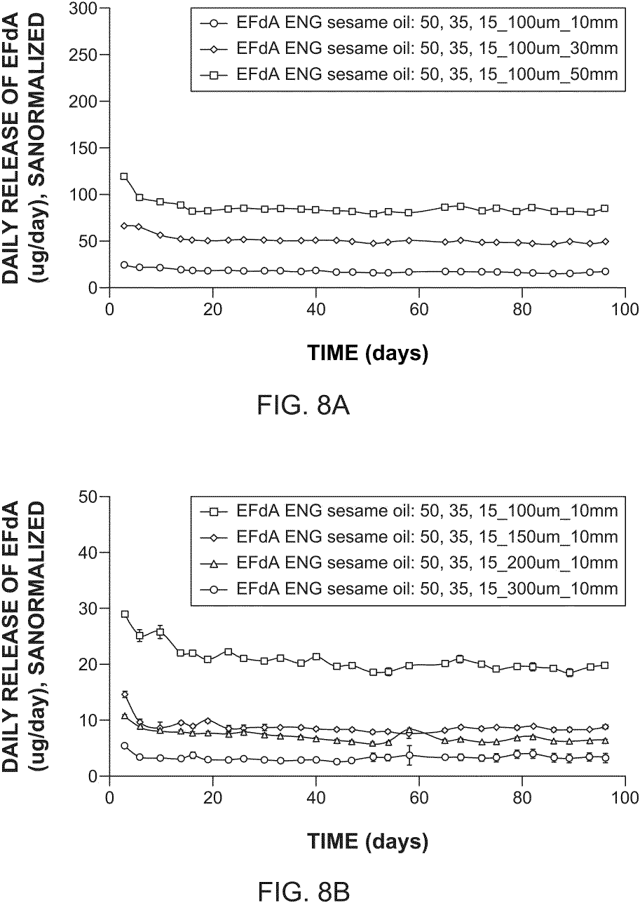

FIG. 8A are line charts showing the daily release profiles of EFdA from EFdA/ENG/Sesame oil formulations at different lengths ranging from 10 to 50 mm. As can be seen, the co-formulated EFdA/ENG/sesame oil devices exhibited linear release profiles with a constant release rate over 90 days. Co-formulated devices with a larger surface area result in a higher release rate of the EFdA.

FIG. 8B are line charts showing the daily release profiles of EFdA from EFdA/ENG/Sesame oil formulations at different wall thicknesses: 100, 150, 200, and 300 µg. Similarly, the release rates of EFdA from the co-formulated EFdA/ENG/sesame oil devices also decrease with increasing wall thickness. This confirms the effect of wall thickness on the release rates of co-formulated devices.

The approximate daily release rates of EFdA formulated with ENG and sesame oil are shown in Table 8. Similarly, the average EFdA release rate of the multi-drug formulations is comparable to the release rates of EFdA alone devices with PC17 extruded tubes. The results of this example further confirm that formulating EFdA with ENG or LNG does not appear to significantly affect the release rate of EFdA.

TABLE 8

THE AVERAGE EFDA RELEASE RATE OF CO-FORMULATED DEVICES CONTAINING EFDA AND ENG FORMULATIONS.

| Formulations | Length of devices | Wall thickness | Release rate of EFdA (µg/day) |
|---|---|---|---|
| EFdA ENG sesame oil: 50, 35, 15 | 10 | 100 | 20.1 ± 2.2 |
| | 30 | 100 | 58.0 ± 5.2 |
| | 50 | 100 | 97.2 ± 8.6 |
| | 10 | 150 | 8.5 ± 1.2 |
| | 10 | 200 | 6.9 ± 1.0 |
| | 10 | 300 | 3.2 ± 0.5 |

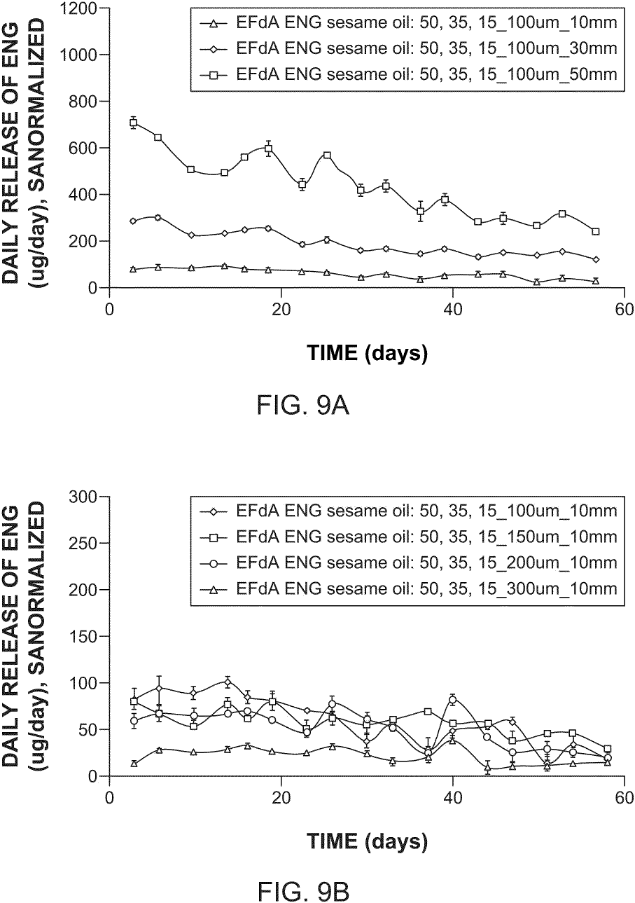

The release of ENG from the EFdA/ENG/sesame oil coformulation was also evaluated. FIGS. 9A and 9B line charts showing daily ENG release profiles of multi-drug devices containing EFdA ENG sesame oil formulations at different lengths over 50 days. Similar to previous data, the release rates of ENG are proportional to the surface area of the implants. Interestingly, unlike the release profiles of EFdA, the release rates of ENG are decreasing over time. This is likely attributed to the depletion of ENG within the device core, as the estimated duration of release for co-formulated EFdA/ENG devices at a wall thickness of 100 µm is ~6 months, whereas the duration of release for the EFdA component is > 1 year.

Taken together, we investigated the effect of wall thickness and surface area on the release of both ARV and hormones from co-formulated devices. As shown, like the single formulations, the release rates of coformulations scale linearly with the surface areas of the implant and are inversely correlated with the wall thickness of the PCL devices. These experiments demonstrate the ability to employ two parameters, surface area or wall thickness, to tailor the release rates of EFdA and hormones from a reservoir-style co-formulated MPT implant.

TABLE 9

THE AVERAGE ENG RELEASE RATE OF CO-FORMULATED DEVICES CONTAINING EFDA AND ENG FORMULATIONS.

| Formulations | Length of devices | Wall thickness | Release rate of ENG (µg/day) |
|---|---|---|---|
| EFdA ENG sesame oil: 50, 35, 15 | 10 | 100 | 51.3 ± 20.2 |
| | 30 | 100 | 162.4 ± 56.6 |
| | 50 | 100 | 256.9 ± 73.1 |
| | 10 | 150 | 48.4 ± 16.72 |
| | 10 | 200 | 44.4 ± 17.5 |
| | 10 | 300 | 28.7 ± 6.6 |

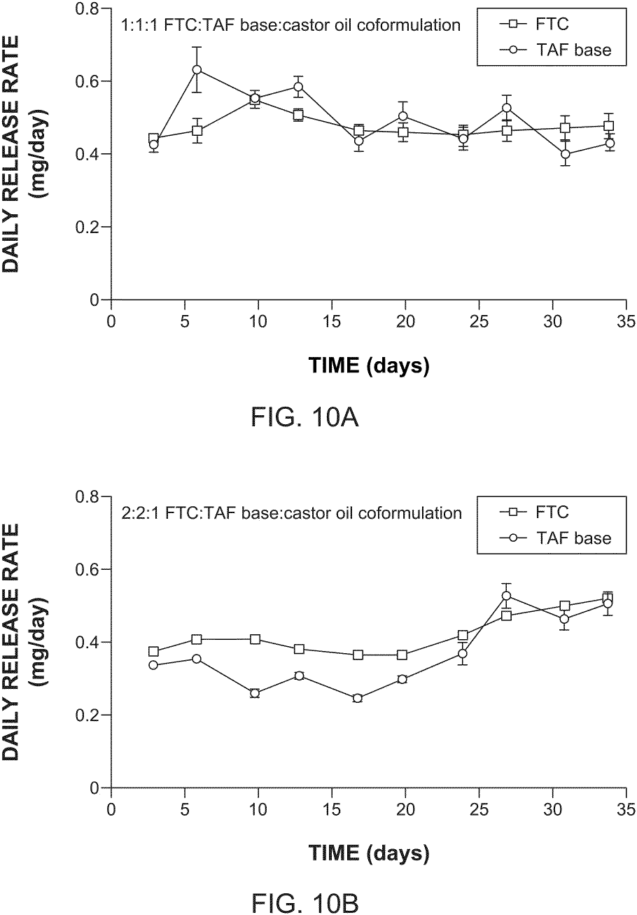

In addition to evaluating ARV/hormone co-formulations, ARVs from the same drug class were co-formulated within the same implant. FIGS. 10A and 10B are line charts showing the daily release profiles of FTC and TAF from 2 different FTC/TAF/castor oil formulations. The PC17 implants had a length of 40 mm, an outer diameter of 2.5 mm, and a wall thickness of 100 µm. As observed, the co-formulated ARV devices exhibited linear release profiles with a constant release rate over 30 days. When the API; excipient ratio was significantly higher, the release profile exhibited a dissolution-controlled mechanism. As explained above, when the dissolution rate is less than the diffusion rate, the release rate is dissolution limited or controlled and the release profile is non-linear.

Table 10 summarizes the overall FTC and TAF release rates from the implants. When the release rate is diffusion-controlled (i.e., 33% FTC formulation), the FTC release rate of the multi-drug formulation was comparable to that of implants containing only FTC and castor oil. Similarly, the TAF release rate from the multi-drug implant was also aligned with previous data wherein implants only had TAF and castor oil. Co-formulating TAF and FTC did not affect the release rate of either drug.

TABLE 10

AVERAGE RELEASE RATE OF CO-FORMULATED DEVICES CONTAINING FTC AND TAF FORMULATIONS.

| API | Excipient | Excipient wt.% | FTC wt.% | TAF wt.% | Release rate of FTC (mg/day/cm) | Release rate of TAF (mg/day/cm) |
|---|---|---|---|---|---|---|
| FTC, TAF | Castor oil | 33 | 33 | 33 | 0.47 ± 0.03 | 0.49 ± 0.07 |
| | | 20 | 40 | 40 | 0.43 ± 0.06 | 0.38 ± 0.09 |

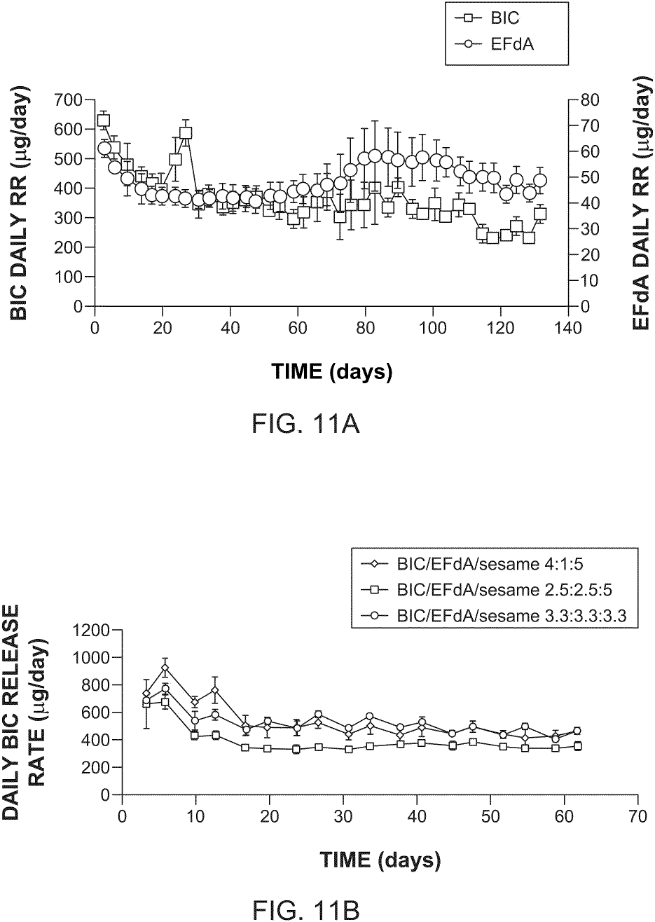

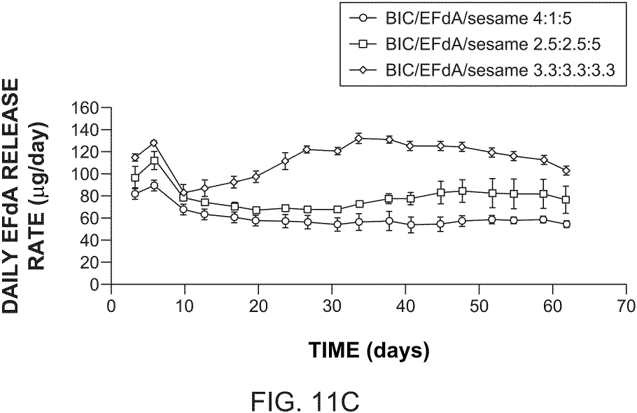

ARVs spanning different drug classes were also co-formulated within the same PC17 implants at a wall thickness of 100 µm and 40 mm length. FIG. 11A is a compilation of line charts showing the daily release profiles of EFdA and BIC from the same implant. Both drugs are formulated at a significantly low API: excipient ratio and did not align with previous data where each of the drugs were individually formulated with the excipient. However, both drugs exhibit linear release profiles up to 130 days.

FIG. 11B shows the linear release profiles up to 60 days of BIC from multi-drug PC17 implants with a wall thickness of 100 µm and device length of 40 mm. As the ratio of BIC within the co-formulation increased, the release rate aligned with that of individual BIC/sesame oil release rate. The release rate is much lower when the BIC component is ≤ 25% within the formulation, which can be attributed to incomplete BIC coverage along the implant length (surface area affects release rate). When there is sufficient amount of BIC present within the implant, its release rate appears to be unaffected in the presence of EFdA.

Release rate of EFdA from multi-drug formulations appears to scale with increases in EFdA ratio within the formulation as observed in FIG. 11C. When the formulation contains 10-25% EFdA, the release rate is aligned with that of implants containing EFdA and sesame oil. As the ratio of EFdA >25%, the presence of BIC appears to affect its release rate.

Table 11 summarizes the release rate of BIC and EFdA across these co-formulations. For both drugs, there appears to be a window in which the release rate of the drug is not affected by the presence of the other within the formulation. Once the amount of either drug falls outside of those limits, the release rates of both BIC and EFdA change depending on their ratio within the formulation.

TABLE 11

AVERAGE RELEASE RATE OF CO-FORMULATED DEVICES CONTAINING BIC AND EFdA FORMULATIONS.

| API | Excipient | Excipient wt.% | BIC wt.% | EFdA wt.% | Release rate of BIC (µg/day/cm) | Release rate of EFdA (µg/day/cm) |
|---|---|---|---|---|---|---|
| BIC, EFdA | Sesame oil | 52.5% | 39.5% | 8% | 361.9 ± 88.0 | 48.5 ± 6.1 |
| | | 50% | 40% | 10% | 538.1 ± 138.3 | 60.9 ± 9.5 |
| | | 50% | 25% | 25% | 395.6 ± 100.9 | 78.8 ± 10.8 |
| | | 33% | 33% | 33% | 527.6 ± 87.1 | 113.2 ± 14.6 |

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

### REFERENCES

(HPTN), H.P.T.N. HPTN 083. A Phase 2b/3 Double Blind Safety and Efficacy Study of Injectable Cabotegravir Compared to Daily Oral Tenofovir Disoproxil Fumarate/Emtricitabine (TDF/FTC), for Pre-Exposure Prophylaxis in HIV-Uninfected Cisgender Men and Transgender Women who have Sex with Men. Available online.

(HPTN), H.P.T.N. HPTN 084. A Phase 3 Double Blind Safety and Efficacy Study of Long-Acting Injectable Cabotegravir Compared to Daily Oral TDF/FTC for Pre-Exposure Prophylaxis in HIV-Uninfected Women Available online.

Solorio, L., Carlson, A., Zhou, H., Exner, A. A. Implantable Drug Delivery Systems. In Engineering Polymer Systems for Improved Drug Delivery, First ed.; Bader, R.A., Putnam, D. A., Ed. John Wiley & Sons, Inc.: 2014; doi:10.1002/9781118747896.ch7.

Yang, W.-W.; Pierstorff, E. Reservoir-Based Polymer Drug Delivery Systems. Journal of Laboratory Automation 2012, 17, 50-58, doi:10.1177/2211068211428189.

Langer, R. Implantable controlled release systems. Pharmacology & Therapeutics 1983, 21, 35-51, doi:https://doi.org/10.1016/0163-7258(83)90066-9.

Gunawardana, M.; Remedios-Chan, M.; Miller, C.S.; Fanter, R.; Yang, F.; Marzinke, M.A.; Hendrix, C.W.; Beliveau, M.; Moss, J.A.; Smith, T.J., et al. Pharmacokinetics of long-acting tenofovir alafenamide (GS-7340) subdermal implant for HIV prophylaxis. Antimicrobial agents and chemotherapy 2015, 59, 3913-3919, doi:10.1128/aac.00656-15.

Chua, C.Y.X.; Jain, P.; Ballerini, A.; Bruno, G.; Hood, R.L.; Gupte, M.; Gao, S.; Di Trani, N.; Susnjar, A.; Shelton, K., et al. Transcutaneously refillable nanofluidic implant achieves sustained level of tenofovir diphosphate for HIV pre-exposure prophylaxis. Journal of Controlled Release 2018, 286, 315-325, doi:https://doi.org/10.1016/j.jconrel.2018.08.010.

A New Collaboration for HIV Prevention, Available online.

Barrett, S.E.; Teller, R.S.; Forster, S.P.; Li, L.; Mackey, M.A.; Skomski, D.; Yang, Z.; Fillgrove, K.L.; Doto, G.J.; Wood, S.L., et al. Extended-Duration MK-8591-Eluting Implant as a Candidate for HIV Treatment and Prevention. Antimicrobial agents and chemotherapy 2018, 62, e01058-01018, doi:10.1128/aac.01058-18.

G. Pitt, C.; Chasalow, F.; Hibionada, Y.M.; M. Klimas, D.; J. Schindler, A. Aliphatic polyesters. I. The degradation of poly(ℇ-caprolactone) in vivo; 1981; Vol. 26, pp. 3779-3787.

Woodruff, M.A.; Hutmacher, D.W. The return of a forgotten polymer— Polycaprolactone in the 21st century. Progress in Polymer Science 2010, 35, 1217-1256, doi:https://doi.org/10.1016/j.progpolymsci.2010.04.002.

1. A reservoir device comprising an active agent formulation contained within a reservoir, wherein the active agent formulation comprises more than one active agent, and wherein the reservoir is defined by a biodegradable, permeable polymer membrane, the membrane allowing for diffusion of the more than one active agent of the formulation there through when positioned subcutaneously in a body of a subject.

2. The device of claim 1, wherein the permeable polymer membrane has a thickness of about 45 µm to about 300 µm, preferably about 70 µm to about 300 µm.

3. The device of claim 1, wherein the active agent formulation comprises the more than one active agent and an excipient.

4. The device of claim 3, wherein at least one of the more than one active agent comprises Tenofovir Alafenamide Fumarate (TAF), 4′-Ethynyl-2-fluoro-2′-deoxyadenosine (EFdA), Abacavir, Levonorgestrel (LNG); Etonogestrel (ENG), emtricitabine (FTC), Tenofovir (TFV), Tenofovir disoproxil fumarate (TDF), EFdAalafenamide, bictegravir, raltegravir, dolutegravir, lamivudine (3TC), tamoxifen citrate, naltrexone or combinations thereof.

5. (canceled)

6. (canceled)

7. (canceled)

8. (canceled)

9. (canceled)

10. (canceled)

11. The device of claim 3, wherein at least one of the more than one active agent comprises an antibody, a small molecule, a protein, a peptide, or a combination thereof.

12. The device of claim 3, wherein the excipient comprises castor oil, sesame oil, oleic acid, polyethylene glycol 600, ethyl oleate, propylene glycol, glycerol, cottonseed oil, polyethylene glycol 40, polyethylene glycol 300, polyethylene glycol 400, Polysorbate 80, Synperonic PE/L 44, α-cyclodextrin, β-cyclodextrin, γ-cyclodextrin or combinations thereof.

13. The device of claim 3, wherein the more than one active agent includes a first active agent and a second active agent, which is different from the first active agent.

14. (canceled)

15. The device of claim 1, wherein the polymer membrane comprises polycaprolactone (PCL), poly(lactic-co-glycolic acid) (PLGA), polylactic acid (PLA) or a blend thereof.

16. The device of claim 1, wherein the polymer membrane comprises polycaprolactone (PCL) at a molecular weight ranging from 15,000-140,000 Da.

17. The device of claim 1, wherein the polymer membrane comprises one or more of a homopolymer, a random co-polymer, an alternating co-polymer, a block co-polymer, a graft co-polymer, a star homopolymer, a star co-polymer.

18. (canceled)

19. (canceled)

20. (canceled)

21. The device of claim 1, wherein the device has a cylindrical shape with a length between about 10 mm and 50 mm.

22. A reservoir device comprising an active agent formulation contained within a reservoir, wherein the active agent formulation comprises more than one active agent, and wherein the reservoir is defined by a biodegradable, permeable polymer membrane, the membrane allowing for diffusion of the more than one active agent there through with zeroorder release kinetics for a time period of at least 60 days when positioned subcutaneously in a body of a subject.

23. The device of claim 22, wherein at least one of the more than one active agent comprises Tenofovir Alafenamide Fumarate (TAF), 4′-Ethynyl-2-fluoro-2′-deoxyadenosine (EFdA), Abacavir, Levonorgestrel (LNG); Etonogestrel (ENG), emtricitabine (FTC), Tenofovir (TFV), Tenofovir disoproxil fumarate (TDF), EFdAalafenamide, bictegravir, raltegravir, dolutegravir, lamivudine (3TC), tamoxifen citrate, naltrexone or combinations thereof.

24. The device of claim 22, wherein at least one of the more than one active agent comprises an antibody, a small molecule, a protein, a peptide, or a combination thereof.

25. The device of claim 22, wherein the reservoir further contains an excipient.

26. (canceled)

27. The device of claim 22, wherein the polymer membrane comprises one or more of a homopolymer, a random co-polymer, an alternating co-polymer, a block co-polymer, a graft co-polymer, a star homopolymer, a star co-polymer.

28. (canceled)

29. The device of claim 22, wherein the polymer membrane comprises polycaprolactone (PCL), poly(lactic-co-glycolic acid) (PLGA), polylactic acid (PLA) or a blend thereof.

30. The device of claim 22, wherein the polymer membrane comprises polycaprolactone (PCL) at a molecular weight ranging from 15,000-140,000 Da.

31. The device of claim 22, wherein the thickness of the permeable polymer membrane is between about 45 µm and about 300 µm.

32. The device of claim 22, wherein the device has a cylindrical shape with a length between about 10 mm and 50 mm.

33. (canceled)

34. The device of claim 1, wherein the polymer membrane is configured such that the membrane undergoes fragmentation at a time ranging from about 1 month to about 6 months after the more than one active agent is depleted from the device.

35. (canceled)

36. (canceled)

37. (canceled)

38. (canceled)

39. (canceled)

40. (canceled)

41. (canceled)

42. A combinatorial method of preventing or aiding in preventing HIV and contraception comprising implanting the device of claim 1 into a subject in need thereof.
